(19) United States
(12) Patent Application Publication
(10) Pub. No.: US 2009/0301476 A1
(43) Pub. Date: Dec. 10, 2009

(54) HEAT AND MOISTURE EXCHANGE UNIT

(76) Inventors: Neil Alex Korneff, Diamond Bar, CA (US); Khalid Said Mansour, Riverside, CA (US); Christopher Jesse Zollinger, Chino Hills, CA (US)

Correspondence Address: DICKE, BILLIG & CZAJA, PLLC;ATTN: CFN MATTERS, 100 SOUTH FIFTH STREET, SUITE 2250, MINNEAPOLIS, MN, 55402, US

(21) Appl. No.: 12/133,958

(22) Filed: Jun. 5, 2008

(51) Int. Cl. A61M 16/00

(52) U.S. Cl. 128/201.13

(57) ABSTRACT

A heat and moisture exchange (HME) unit including a housing, a heat and moisture retaining media (HM media), and a valve mechanism. The housing forms an intermediate section extending between two ports, and defining first and second flow paths. The HM media is maintained along the first flow path. The valve mechanism includes an obstruction member movably retained within the housing and transitionable between opposing, first and second maximum points of travel. At the first maximum point of travel, the obstruction member closes the second flow path to permit airflow through only the first flow path. At the second maximum point of travel, the obstruction member permits airflow through both of the first and second flow paths. The HME unit is simple to use, yet provides an effective bypass state in which airflow freely progresses around the HM media.

### BACKGROUND

The present disclosure relates to a heat and moisture exchange (“HME”) unit useful with a patient breathing circuit. More particularly, the HME unit of the present disclosure is connectable to a breathing circuit and provides a bypass construction selectively enabling air flow to pass through the HME unit with minimal interaction with a contained heat and moisture retaining media.

The use of ventilators and breathing circuits to assist in patient breathing is well known in the art. The ventilator and breathing circuit provide mechanical assistance to patients who are having difficulty breathing on their own. During surgery and other medical procedures, the patient is often connected to a ventilator to provide respiratory gases to the patient. One disadvantage of such breathing circuits is that the delivered air does not have a humidity level and/or temperature appropriate for the patient's lungs.

In order to provide air with desired humidity and/or temperature to the patient, an HME unit can be fluidly connected to the breathing circuit. As a point of reference, “HME” is a generic term, and can include simple condenser humidifiers, hygroscopic condenser humidifiers, hydrophobic condenser humidifiers, etc. In general terms, HME units consist of a housing that contains a layer of heat and moisture retaining media or material (“HM media”). This material has the capacity to retain moisture and heat from the air that is exhaled from the patient's lungs, and then transfer the captured moisture and heat to the ventilator-provided air of the inhaled breath. The HM media can be formed of foam or paper or other suitable materials that are untreated or treated, for example, with hygroscopic material.

While the HME unit addresses the heat and humidity concerns associated with ventilator-provided air in a breathing circuit, other drawbacks may exist. For example, it is fairly common to introduce aerosolized medication particles into the breathing circuit (e.g., via a nebulizer) for delivery to the patient's lungs. Where an HME unit is present in the breathing circuit, however, the medication particles will not readily traverse through the HM media and thus not be delivered to the patient. In addition, the HM media can become clogged with the droplets of liquid medication, in some instances leading to an elevated resistance of the HME unit. One approach for addressing these concerns is to remove the HME unit from the breathing circuit when introducing aerosolized medication. This is time consuming and subject to errors, and can result in the loss of recruited lung volume when the circuit is depressurized. Alternatively, various HME units have been suggested that incorporate intricate bypass structures/valves that selectively and completely isolate the HM media from the airflow path. For example, existing bypass-type HME units employ a bypass structure that is internal or through the HM media. While viable, these and other bypass-type HME units are difficult to operate (e.g., requiring a caregiver to rotate two, frictionally fitted housing units relative to one another) and/or are relatively complex and thus expensive.

In light of the above, a need exists for improved HME units having an HM media bypass feature that addresses one or more of the problems associated with conventional bypass-type HME units.

### SUMMARY

Some aspects in accordance with the present disclosure relate to a heat and moisture exchange (HME) unit including a housing, a heat and moisture retaining media (HM media), and a valve mechanism. The housing forms a first port, a second port, and an intermediate section. The intermediate section extends between the first and second ports, and defines first and second flow paths fluidly connecting the first and second ports. The HM media is maintained within the intermediate section along the first flow path. The valve mechanism includes an obstruction member movably retained within the housing and transitionable between opposing, first and second maximum points of travel. In this regard, the HME unit is configured such that at the first maximum point of travel, the obstruction member closes the second flow path to permit airflow through only the first flow path. At the second maximum point of travel, the obstruction member permits airflow through both of the first and second flow paths. With this construction, the HME unit is compact and simple to use, yet provides an effective bypass state in which airflow freely progresses around the HM media. In some embodiments, the first flow path is U-shaped in cross-section, with the HM media arranged in the first flow path such that airflow is through opposing major surfaces of the HM media. In yet other embodiments, the HME unit further includes a check valve plate arranged to permit airflow through the second flow path in a first flow direction and prevent airflow through the second flow path in a second, opposite flow direction.

Other aspects in accordance with principles of the present disclosure relate to an HME unit including a housing, a HM media, and a valve mechanism. The housing includes an intermediate section extended between first and second ports. The HM media defines opposing, first and second sides, and is disposed within the housing such that the first side fluidly faces the first port and the second side fluidly faces the second port. The valve mechanism includes an obstruction member movably assembled within the intermediate section of the housing, fluidly between the first side of the HM media and the first port. In this regard, the obstruction member is transitionable from an HME position in which the obstruction member completes a flow path from the first port, through the HM media, and to the second port, and closes a bypass flow path around the HM media. Further, the HME unit is configured such that in any position of the obstruction member relative to the housing, at least a portion of the first side of the heat and moisture media remains is fluidly open to the first port. With this construction, the HME unit can have a compact construction yet provide an effective bypass state in which airflow freely travels around the HM media.

Yet other aspects in accordance with the present disclosure relate to an HME unit including a housing, an HM media, a secondary filter, and a valve mechanism. The housing forms an intermediate section extending between first and second ports, with the intermediate section forming first and second flow paths. The HM media and the secondary filter are maintained along the first flow path, apart from the second flow path. The valve mechanism includes an obstruction member movably assembled within the housing and transitionable between first and second positions. In the first position, the first flow path is open and the second flow path is closed. In the second position, at least the second flow path is open. With this configuration, the HME unit can serve as an HMEF, with the secondary filter being relatively large that in turn results in a higher filter efficiency as compared to convention, bypass-type HMEF units.

### BRIEF DESCRIPTION OF THE DRAWINGS

FIG. 1 is a simplified illustration of an example patient breathing circuit with which an HME unit in accordance with principles of the present disclosure is useful;

FIG. 2 is a simplified illustration of another example breathing circuit with which the HME unit in accordance with principles of the present disclosure is useful;

FIG. 3A is a perspective view of an HME unit in accordance with principles of the present disclosure in an HME mode;

FIG. 3B is a perspective view of the HME unit of FIG. 3A in a bypass mode;

FIG. 4A is a longitudinal cross-sectional view of the HME unit of FIG. 3A;

FIG. 4B is a longitudinal cross-sectional view of the HME unit of FIG. 3B;

FIG. 4C is a lateral cross-sectional view of the HME unit of FIG. 3A;

FIG. 5 is a lateral cross-sectional view of the HME unit of FIG. 3A, illustrating an optional resistance indicator;

FIG. 6 is a top, side perspective view of another HME unit in accordance with principles of the present disclosure

FIG. 7A is a longitudinal, cross-sectional view of the HME unit of FIG. 6 in an HME mode;

FIG. 7B is a longitudinal, cross-sectional view of the HME unit of FIG. 6 in a bypass mode;

FIGS. 8 and 9 are cross-sectional views illustrating portions of another HME unit in accordance with principles of the present disclosure;

FIGS. 10 and 11 are cross-sectional views illustrating portions of another HME unit in accordance with principles of the present disclosure;

FIGS. 12 and 13 are simplified cross-sectional views illustrating portions of another HME unit in accordance with principles of the present disclosure; and

FIGS. 14 and 15 are simplified cross-sectional views illustrating portions of another HME unit in accordance with principles of the present disclosure.

### DETAILED DESCRIPTION

As described in detail below, aspects in accordance with principles of the present disclosure relate to an HME unit useful with a patient breathing circuit. As a point of reference, FIG. 1 illustrates one such breathing circuit 10 as including a number of flexible tubing segments that are connected in between a patient 12 and a ventilator (not shown). The breathing circuit 10 of FIG. 1 is a dual limb breathing circuit, and can include a source of pressurized air 14, an HME unit 16 (shown in block form) in accordance with the present disclosure, and a nebulizer 18.

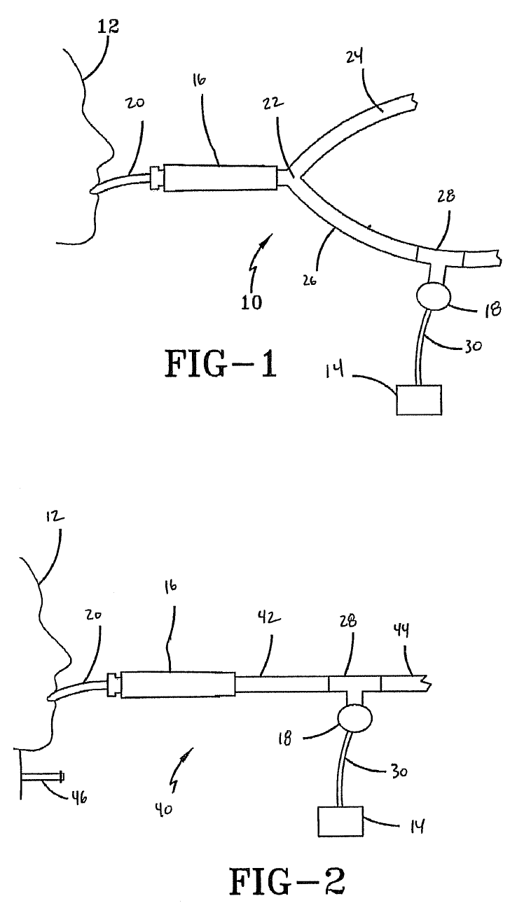

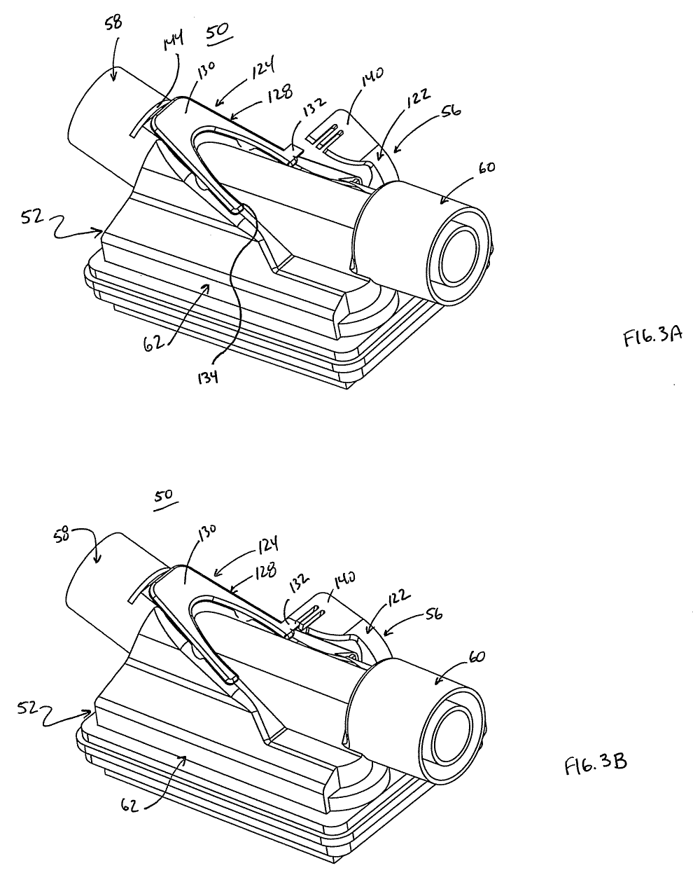

With the one, non-limiting example of the breathing circuit 10 in mind, a patient tube 20 is provided that connects the patient 12 to the HME unit 16. An end of the patient tube 20 that interfaces with the patient 12 can be an endrotracheal tube that extends through the patient's mouth and throat and into the patient's lungs. Alternatively, it also may be connected to a tracheotomy tube (not shown in FIG. 1, but referenced at 46 in FIG. 2) that provides air to the patient's throat and thereby to the patient's lungs. Extending on an opposite side of the HME unit 16 is a connector 22, for example a Y-connector. The Y-connector 22 can be connected to additional tubing for example, an exhalation tube 24 (commonly referred to as the “exhalation limb”) that allows exhaled air to leave the breathing circuit 10. A second tube 26 (commonly referred to as the “inhalation limb”) can serve as a nebulizer tube, and is connected to the nebulizer 18. The nebulizer 18, in turn, is connected to the inhalation limb 26 via a connector 28, for example a T-connector. The T-connector 28 is connected at an end opposite the inhalation limb 26 to a ventilator (not shown). The nebulizer 18, in turn, is also connected to the source of pressurized air 14 via an air tube 30.

By way of further reference, FIG. 2 illustrates an alternative breathing circuit 40 with which the HME unit 16 of the present disclosure is useful. The breathing circuit 40 is a single limb breathing circuit that again serves to fluidly connect a ventilator (not shown) with the patient 12, and includes the nebulizer 18 and the source of pressurized air 14. With the single limb breathing circuit 40, the patient tube 20 is again provided, fluidly connecting the patient 12 and the HME unit 16. A single tube 42 extends from the HME unit 16 opposite the patient 12, and is fluidly connected to the nebulizer 18 via the T-connector 28. The ventilator (not shown) is directly connected to the T-connector 28 via a tube 44. Where desired, the single limb breathing circuit 40 (as well as the dual limb breathing circuit 10 of FIG. 1) can be connected to a tracheotomy tube 46.

The present disclosure contemplates use of various types of nebulizers 18. With one example nebulizer 18, medication is provided which has been reconstituted with sterile water and placed in a reservoir provided in the nebulizer 18. Pressurized gas is provided to the nebulizer 18 that is blown across an atomizer within the nebulizer 18. The force of the gas over the atomizer pulls the medicated liquid from the medication reservoir up along the sides of the nebulizer 18 in a capillary action to provide a stream of the medicated liquid at the atomizer. When the medicated liquid hits the stream of forced air at the atomizer, the liquid is atomized into a multiplicity of small droplets. The force of the air propels this now nebulized mixture of air and medicated liquid into the breathing circuit 10, 40 and to the patient 12, where the medication is provided to the patient's lungs. Use of administration of medication in this procedure has been found to be highly effective in providing the medication through the lungs to the patient. Metered dose inhalers can also be used to provide medication in the air to the patient 12.

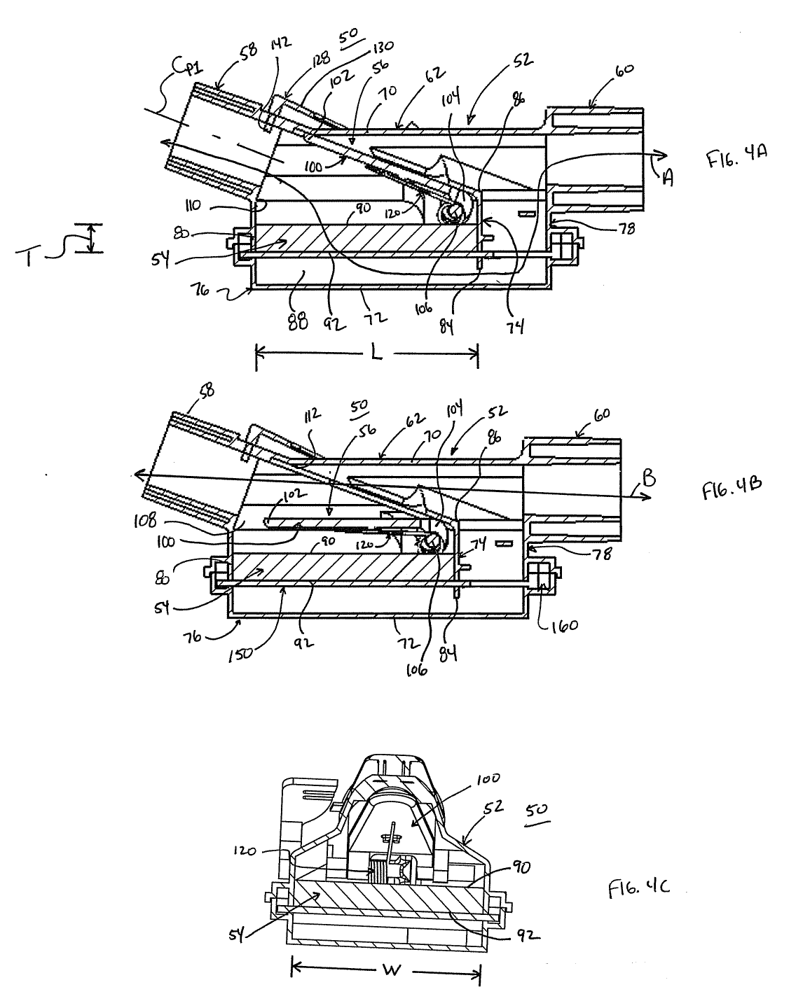

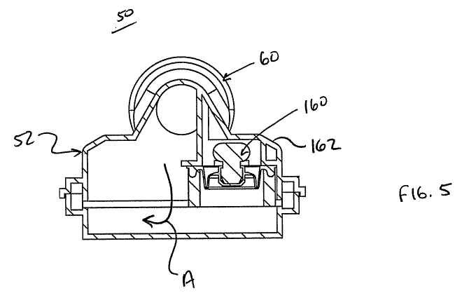

With the above general explanation of breathing circuits in mind, one configuration of an HME unit 50 useful as the HME unit 16 (FIGS. 1 and 2) is shown in FIGS. 3A and 3B. The HME unit 50 includes a housing 52, a heat and moisture media (HM media) 54 (hidden in FIGS. 3A and 3B, but shown in FIG. 4A), and a valve mechanism 56 (referenced generally). Details on the various components are provided below. In general terms, however, the housing 52 forms a first port 58, a second port 60, and an intermediate section 62. The HM media 54 is retained within the intermediate section 62. The housing 52 generally defines flow paths fluidly connecting the ports 58, 60, including a first flow path through the HM media 54, and a second flow path around (e.g., to the side of) the HM media 54. In this regard, the valve mechanism 56 is operable to dictate the pathway through which airflow will at least primarily occur. As a point of reference, FIG. 3A depicts the HME unit 50 in an HME mode, and FIG. 3B depicts the HME unit 50 in a bypass mode as described below.

The housing 52, including the flow paths formed thereby, is further illustrated in FIGS. 4A and 4B (with FIG. 4A depicting the HME mode of FIG. 3A and FIG. 4B depicting the bypass mode of FIG. 3B). As shown, the intermediate section 62 extends between the first and second ports 58, 60. Relative to the upright orientation of FIGS. 4A and 4B, the intermediate section 62 forms an upper exterior portion or wall 70, a lower exterior portion or wall 72, and at least one interior partition 74. With some configurations, the lower wall 72 is provided as part of a first housing segment 76 that is removably mounted to a second housing segment 78 that otherwise provides the ports 58, 60 and the upper wall 70. Regardless, the HM media 54 is retained within the intermediate section 62, for example nested between the exterior partition 74 and a side wall 80. One or more other components can assist in maintaining the HM media 54 at a desired location relative to the interior partition 74 and the valve mechanism 56. Further, the interior partition 74 is a solid body defining, at least in part, a first flow path (designated in FIG. 4A by an arrow “A”) and a second flow path (designated in FIG. 4B by an arrow “B”). More particularly, the interior partition 74 forms opposing first and second ends 84, 86, with the first flow path A being formed, in part, between the first end 84 and the lower wall 72, and the second flow path B being formed, in part, between the second end 86 and the upper wall 70.

The first flow path A progresses from the first port 58, through the HM media 54, and to the second port 60 (and vice-versa), and thus can be referred to as an HME pathway. With the one configuration of FIG. 4A, the HM media 54 is sized and positioned within the housing 52 such that a gap 88 is formed between the HM media 54 and the lower wall 72, with the first flow path A traversing through the gap 88 and around the first end 84 of the interior partition 74 to establish a U-shaped pathway. With other configurations, however, the HM media 54 can be in contact with the lower wall 72 (or the gap 88 otherwise eliminated).

The second flow path B progresses from the first port 58, through the intermediate section 62, and to the second port 60 (and vice-versa), and does not include the HM media 54. Thus, the second flow path B can be referred to as a bypass pathway. The bypass pathway B is around, or to the side of, the HM media 54. Unlike conventional bypass-type HME units, the bypass pathway (i.e., the second flow path B) in accordance with some aspects of the present disclosure does not go “through” the HM media 54, and thus enables implementation of more user-friendly valving configurations as described below.

As indicated above, the HM media 54 is sized and shaped for placement within the intermediate section 62. In this regard, the HM media 54 can assume a variety of forms known in the art that provide heat and moisture retention characteristics, and typically is or includes a foam material. Other configurations are also acceptable, such as paper or filler-type bodies. In more general terms, then, the HM media 54 can be any material capable of retaining heat and moisture regardless of whether such material is employed for other functions (e.g., filtering particle(s)). With some constructions, the HM media 54 has a generally rectangular shape, defining opposing, first and second major surfaces 90, 92. Upon final assembly, the HM media 54 is arranged such that the first major surface 90 fluidly faces the first port 58, whereas the second major face 92 fluidly faces the second port 60. In other words, relative to the first flow path A, the first major surface 90 serves as the face at which airflow from the first port 58 initially interacts with the HM media 54 (and vice-versa); similarly, airflow from the second port 60 along the first flow path A initially interfaces with the second major surface 92 (and vice-versa). With these designations in mind, and with additional reference to FIG. 4C, the HME unit 50 orients the HM media 54 such that a relatively large HM media surface area (i.e., the first or second major surface 90, 92) is presented within the first flow path A, yet overt airflow restrictions are minimized. More particularly, flow along the first airflow path A progresses through a thickness T of the HM media 54, with the thickness T being less than a length L or width W (FIG. 4C) of the HM media 54. In some embodiments, the gap 88 (otherwise provided fluidly adjacent the second major surface 92) facilitates this desirable airflow characteristic. As such, resistance to normal patient breathing through the HME unit 50 is minimized.

As indicated above, the valve mechanism 56 dictates which of the flow paths A or B airflow between the ports 58, 60 will at least primarily occur. In this regard, the valve mechanism 56 includes an airflow obstruction member 100 that is movably disposed or assembled within the intermediate section 62 as best shown in FIGS. 4A and 4B. Other components associated with some constructions of the valve mechanism 56 in accordance with the present disclosure are described below. Further, the obstruction member 100 can assume a variety of shapes, and is generally provided as a solid body or bodies through which airflow cannot pass. In the one configuration of FIGS. 4A and 4B, the obstruction member 100 is plate-like; alternatively, other valving obstruction bodies (e.g., ball valve, etc.) are also acceptable. Regardless, the obstruction member 100 is transitionable between a first position shown in FIG. 4A and a second position shown in FIG. 4B. For example, with the one configuration of FIGS. 4A and 4B, the obstruction member 100 is akin to a plate, defined by a leading end 102 and a trailing end 104. The trailing end 104 is pivotably mounted within the housing 52, for example, via a pin 106. Other transitionable assembly constructions are also acceptable, such as by providing the trailing end 104 as a living hinge. With these constructions, then, transitioning of the obstruction member 100 includes the obstruction member 100 pivoting at the trailing end 104, with the leading end 102 traveling between the first and second positions. With this in mind, the leading end 102 is configured to engage or seal against a corresponding structure of the housing 52, for example, the upper wall 70, in the first position of FIG. 4A. In other words, the obstruction member 100 is sized and shaped such that in the first position, the obstruction member 100 closes the second flow path B, thereby forcing or dictating that all airflow occur along the first flow path A. Because the first flow path A includes the HM media 54, the first position of the obstruction member 100 can be referred to as an “HME position” or “HME mode”.

With some embodiments, the housing 52 and the valve mechanism 56 are configured to create a more streamlined pathway between the first port 58 and the HM media 54 in the HME position. For example, the first port 58 defines a central axis C_{P1}. In the first position of the obstruction member 100, the obstruction member 100 is arranged such that a major plane thereof is substantially parallel with the central axis C_{P1} of the first port 58, thereby directing airflow directly toward the HM media 54 (and vice-versa). That is to say, in the first position of the obstruction member 100, airflow does not encounter a 90 degree corner between the first port 58 and the HM media 54. Alternatively, other relationships between the first port 58 and the obstruction member 100 can be established.

With specific reference to FIG. 4B, in the second position of the obstruction member 100, the leading end 102 is transitioned (e.g., pivoted at the trailing end 104) away from engagement with the upper wall 70, such that the second flow path B is not obstructed by the obstruction member 100. Notably, in the second position, the obstruction member 100 does not completely obstruct or close the first flow path A. For example, a spacing 108 exists between the leading end 102 of the obstruction member 100 and the corresponding side wall 80 of the housing 52. Stated otherwise, the housing 52 effectively creates a first passageway opening 110 relative to the HM media 54 along the first flow path A (e.g., defined between the interior partition 74 and the side wall 80), and a second passageway opening 112 relative to the second flow path B. The obstruction member 100 has a size and a shape commensurate with those of the second passageway opening 112, and thus extends across and closes the second passageway opening 112 in the second position. However, a size and a shape of the obstruction member 100 is less than those of the first passageway opening 110 such that the obstruction member 100 cannot encompass an entirety of the first passageway opening 110. In other words, regardless of a position of the obstruction member 100 relative to the housing 52, the obstruction member 100 never completely closes the first flow path A.

In the second position of the obstruction member 100, the second flow path B is at most only partially obstructed by the obstruction member 100, thereby allowing airflow to freely progress to and from the first and second ports 58, 60 without intimately encountering the HM media 54. Thus, the second position of the obstruction member 100 can be referred to as a “bypass position” or “bypass mode”. In the bypass position, airflow can still occur along the first flow path A via the spacing 108. However, the HM media 54 effectively serves to restrict or resist airflow through the spacing 108. In particular, because airflow will seek the path of least resistance, in the bypass position of the obstruction member 100, a vast majority of the airflow will occur directly through or along the second flow path B. In fact, it has surprisingly been found that at least 95%, in other embodiments at least 97%, and in yet other embodiments at least 98%, of airflow will occur through the second flow path B with the obstruction member 100 in the bypass position as described below.

As a point of reference, the first position (FIG. 4A) and the second position (FIG. 4B) of the obstruction member 100 as described above reflect opposite, first and second maximum points of travel of the obstruction member 100. For example, the first maximum point of travel is established by leading end 102 (or other portion of the obstruction member 100) contacting the upper wall 70 such that the upper wall 70 prevents movement (e.g., clockwise rotation relative to the orientation of FIG. 4A) of the obstruction member 100 beyond the first position. Conversely, the housing 52 can include a stop or other feature that prevents movement (e.g., counterclockwise rotation relative to the orientation of FIG. 4B) of the obstruction member 100 beyond the second position, thus defining the second maximum point of travel. Alternatively, the HME unit 50 can be configured to establish the second maximum point of travel as differing from that of FIG. 4B (e.g., the second position can include the obstruction member 100 contacting the HM media 54). In accordance with principles of the present disclosure, however, at no position of the obstruction member 100 relative to the housing 52 does the obstruction member 100 completely obstruct or close the first flow path A.

In some embodiments, the valve mechanism 56 is configured to permit a user to manually effectuate transitioning and locking of the obstruction member 100 to the desired position or mode. For example, in some embodiments, the valve mechanism 56 includes a biasing member 120, such as a torsional spring, that biases the obstruction member 100 to the first or HME position. This arrangement simplifies the bypass mechanism and assists in ensuring seal integrity independent of operator interaction/use (e.g., the operator is not required guess as to whether the HME position has been achieved). With additional reference to FIGS. 3A and 3B, the valve mechanism 56 can further include an actuator arm 122 and an optional release device 124, both of which are accessible at an exterior of the housing 52. The actuator arm 122 is coupled to the obstruction member 100 (for example, via the pin 106), such that the obstruction member 100 moves (e.g., pivots) with movement of the actuator arm 122. Thus, a rotational position of the actuator arm 122 in FIG. 3A corresponds with the first position of the obstruction member 100 illustrated in FIG. 4A. Conversely, a rotational position of the actuator arm 122 in FIG. 3B corresponds with the second position (bypass position) of the obstruction member 100 shown in FIG. 4B. Thus, to transition the HME unit 50 from the HME mode (FIGS. 3A and 4A) to the bypass mode (FIGS. 3B and 4B), a user (not shown) applies a moment force onto the actuator arm 122 sufficient to overcome a spring force or bias of the spring 120, thereby causing the obstruction member 100 to pivot or rotate from the HME position to the bypass position.

One or more features can be included for selectively capturing and holding one or both of the obstruction member 100 and/or the actuator arm 122 in the bypass position. For example, the release device 124 can be configured to releasably engage the actuator arm 122 as described below. Further, where provided, the optional release device 124 is operable to selectively unlock the actuator arm 122, thus the obstruction member 100, from the bypass position. As best shown in FIGS. 3A and 3B, the release device 124 can include a switch member 128 movably (e.g., pivotably) mounted to the housing 52. The switch member 128 forms a contact surface 130 and an engagement finger 132. The contact surface 130 is sized to readily receive a user's finger in manipulating or operating the release device 124, and is formed or provided opposite a point of attachment 134 of the switch member 128 relative to the housing 52. The engagement finger 132 is sized to selectively abut or interface with a corresponding feature of the actuator arm 122. For example, in some embodiments, the actuator arm 122 includes or forms a head 140. With these conventions in mind, the actuator arm 122 and the switch member 128 are configured and arranged such that upon final assembly, the head 140 (or other component associated with the head 140 such as a latch) contacts the engagement finger 132 as the actuator arm 122 is rotated to the bypass mode or position (FIGS. 3B and 4B). In some embodiments, the engagement finger 132 and the head 140 are captured relative to one another in the bypass position or mode, thereby effectuating a temporary lock. Regardless, the valve mechanism 56 can be released from the bypass position or mode by a user applying a force onto the contact surface 130. This so-applied force causes the switch member 128 to rotate about the point of attachment 134, in turn causing the engagement finger 132 to force (or release) the head 140, and thus the actuator arm 122, away from the locked engagement in the bypass mode or position. Alternatively or additionally, a pulling force can be applied by the user on to the head 140 to cause disengagement from the switch member 128 (or other locking device where provided). Once released, the biasing member 120 biases or forces the obstruction member 100 to the HME position.

The HME unit 50 can include one or more additional features that facilitate the above-described movement of the switch member 128. For example, the switch member 128 can further include a shoulder 142 (illustrated in FIG. 4A) extending opposite the contact surface 130, with the housing 52 (e.g., the first port 58) forming a slot 144 (FIG. 3A) sized to slidably receive the shoulder 142. With this construction, then, the shoulder 142/slot 144 interface permits and guides movement of the switch member 128 in releasing the actuator arm 122 from the locked, bypass position. Alternatively, the shoulder 142 and the slot 144 can be eliminated. The valve mechanism 56 can incorporate other components adapted to facilitate manual transitioning and locking of the obstruction member 100 to the desired position or mode as described below.

The HME unit 50 can include one or more additional, optional features. For example, and with reference to FIG. 4B, the housing segments 76, 78 can be separately formed and selectively assembled to one another. With this construction, the HM media 54 can be readily accessed and replaced by simply removing the first housing segment 76 from the second housing segment 78. Further, a secondary filter 150 can be provided. The secondary filter 150 can assume a variety of forms (e.g., HMEF as known in the art), and is assembled directly adjacent the HM media 54. With the one construction of FIG. 4B, the secondary filter 150 abuts the second major surface 92 of the HM media 54, and thus can have a relatively large filtration surface area commensurate with a surface area of the HM media 54. Further, the bypass features of the HME unit 50 described above with respect to the HM media 54 are equally applicable relative to the secondary filter 150. Thus, the secondary filter 150 can be bypassed in the identical manner as the HM media 54. As compared to previous HME devices that either do not include a secondary filter or provide the filter apart from the HM media bypass features, the secondary filter 150 in accordance with the present disclosure can be relatively large, enabling lower resistance and higher filtration efficiency. As a point of reference, FIG. 4B illustrates the secondary filter 150 as being captured between walls formed by the housing segments 76, 78, and as supporting the HM media 54 upon final assembly. The secondary filter 150 is an optional component in accordance with the present disclosure, and it will be understood that the HM media 54 can provide desired filtering in and of itself.

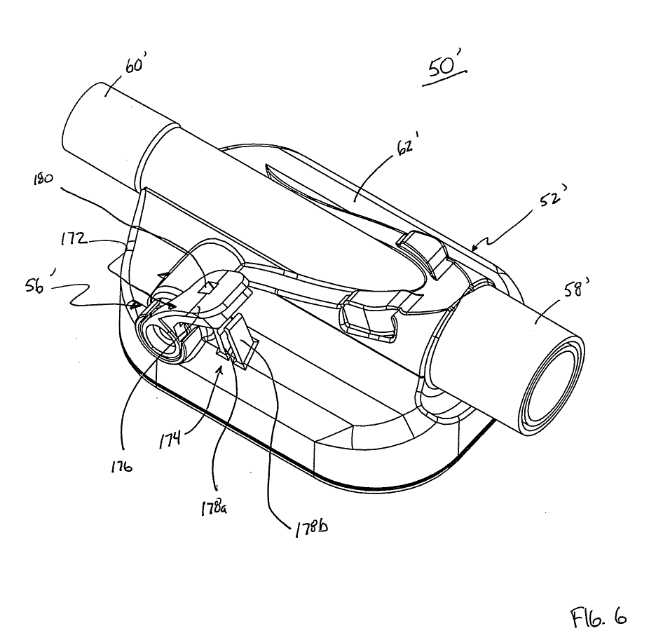

An additional, optional feature provided with the HME unit 50 is a resistance indicator 160. The resistance indicator 160 can assume a variety of forms, and generally serves to identify instances where a differential pressure or resistance across the HME unit 50 (in the HME mode) has exceeded a predetermined value. For example, as shown in FIG. 5, the resistance indicator 160 is in fluid communication with the second port 60 along the first flow path A, and is thus exposed to an internal pressure differential within the HME unit 50 across the HM media 54 (FIG. 4A) relative to the second port 60. The resistance indicator 160 can be mechanical (e.g., silicone diaphragm) and/or incorporate electronic components. Regardless, when triggered (i.e., in the presence of an excessive pressure differential across the HM media 54), the resistance indicator 160 provides a warning or other indication to a caregiver of a potentially problematic state of the HME unit 50 (e.g., the HM media 54 is overtly resisting airflow). In this regard, where the resistance indicator 160 is internally disposed within the housing 52, one or more exterior walls 162 associated with the housing 52 and located in close proximity to the resistance indicator 160 can be at least partially transparent such that the resistance indicator 160 is viewable through the housing 52. In other embodiments, the resistance indicator 160 can be omitted.

Returning to FIGS. 3A-4B, an additional, optional feature provided with the HME unit 50 is a check valve (not shown). The check valve is provided apart from the valve mechanism 56, and is described in greater detail below with respect to other HME unit embodiments. In general terms, however, the check valve is movably retained within the housing 52 along the second flow path B, and is configured to permit airflow through the second flow path B in a first flow direction (e.g., from the second port 60 to the first port 58) and prevent airflow through the second flow path B in a second, opposite flow direction (e.g., from the first port 58 to the second port 60).

Regardless of whether one or more of the optional features described above are provided, during use the HME unit 50 is fluidly connected to a patient breathing circuit, for example the breathing circuit 10 of FIG. 1 or the breathing circuit 40 of FIG. 2. The patient tube 20 is fluidly connected to the first port 58, and the second port 60 is fluidly connected to tubing connected to the ventilator (not shown). Thus, the first port 58 serves as a patient-side port, and the second port 60 serves as a ventilator-side port. In instances where medication is not being provided to the patient 12 via the breathing circuit 10, 40 (i.e., the nebulizer 18 is either not connected to the breathing circuit 10, 40 and/or is non-operational), the HME unit 50 is operated in the HME mode (FIGS. 3A and 4A). Thus, airflow to and from the patient 12 via the HME unit 50 must pass through the HM media 54 (as well as the optional secondary filter 150 where provided), with the HM media 54 absorbing moisture and heat from exhaled air, and then transferring moisture and heat to the inhaled air provided to the patient's lungs.

In instances where the nebulizer 18 is operated to administer nebulized medication to the patient 12, the HME unit 50 is readily transitioned from the HME mode to the bypass mode (FIGS. 3B and 4B) by a user pressing on the actuator arm 122. With the obstruction member 100 in the bypass position, airflow to and from the patient 12, via the HME unit 50, occurs primarily along the bypass flow path B (due to the resistance created by the HM media 54), and thus around (e.g., to the side of) the HM media 54 (as well as the optional secondary filter 150 where provided). In the bypass mode, then, the possibility of the HM media 54 becoming clogged with medication droplets is virtually eliminated.

The ability of the HME unit 50 to desirably function as a bypass-HME has been confirmed through testing. More particularly, a non-limiting example HME unit in accordance with FIGS. 4A-4C was constructed to include an HM media formed of polyurethane foam, and having a length L of 2.75 inches, a width W of 2.0 inches, and a thickness T of 0.375 inch. A secondary filter (i.e., the secondary filter 150) was further included in the form of polypropylene fibers, having a length and width commensurate with the HM media, and a thickness of 0.050 inch. Finally, the housing and obstruction member were construed such that in the closed or bypass position, a gap (i.e., the gap 108) was 0.150 inch. The so-constructed HME unit was then tested by forcing airflow into the first or patient-side port (i.e., the port 58), and a flow rate along the HM flow path (i.e., the HM flow path A) was measured adjacent the HM media, opposite the patient-side port (i.e., a port formed along the lower wall 72). Under these conditions, it was surprisingly found that at an air source flow rate of 30 liters/minute, approximately 0.74% of the flow penetrated through the HM media/secondary filter (with the obstruction member in the closed or bypass position). At an air source flow rate of 60 liters/minute, approximately 1.13% of the flow penetrated through the HM media/secondary filter. Finally, at a maximum air source flow rate of 77.5 liters/minute, approximately 1.25% of the flow penetrated through the HM media/secondary filter. Thus it was surprisingly confirmed that even through a complete flow isolation of the HM media in the bypass state was not provided, the HME unit fully functioned as a bypass HME unit.

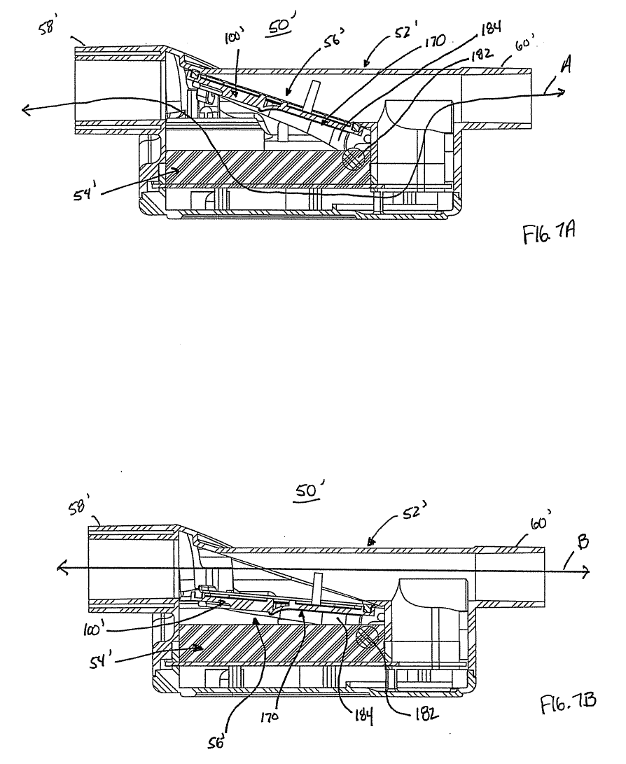

The HME unit 50 described above is but one acceptable configuration in accordance with principles of the present disclosure. For example, a related embodiment HME unit 50′ in accordance with principles of the present disclosure is shown in FIG. 6 and includes a housing 52′, an HM media 54′ (hidden in FIG. 6, but shown in FIGS. 7A and 7B), and a valve mechanism 56′ (referenced generally). The housing 52′ forms a first port 58′ (e.g., a patient side port), a second port 60′ (e.g., ventilator side port), and an intermediate section 62′. The HM media 54′ is retained within the intermediate section 62′, with the valve mechanism 56′ operating to dictate a pathway through which airflow at least primarily progresses between the first and second ports 58′, 60′ as described below.

In particular, and with reference to FIGS. 7A and 7B, the housing 52′ defines first and second flow paths between the ports 58′, 60′, as designated by an arrow A in FIG. 7A and an arrow B in FIG. 7B. The first flow path A includes the HM media 54′, whereas the second flow path B does not. In other words, air flowing through the first path A interacts with the HM media 54′ and thus constitutes an HME pathway. Conversely, air flowing in the second flow path B does not intimately interact with the HM media 54′, and thus serves as a bypass pathway. As with previous embodiments, the second flow path/bypass pathway B is around (e.g., to the side of) the HM media 54′. As a point of reference, FIGS. 7A and 7B illustrate an even more streamlined pathway (as compared to the HME unit 50 described above) along the second flow path/bypass pathway B whereby the central axes of the first and second ports 58′, 60′ are parallel, and in some embodiments, co-axial.

The valve mechanism 56′ includes an obstruction member 100′ that is movably assembled within the housing 52′ as described above. As compared with the HME unit 50 (FIG. 1), however, the valve mechanism 56′ incorporates a differing construction for effectuating manual transition of the obstruction member 100′ between the HME position of FIG. 7A and the bypass position of FIG. 7B. For example, the valve mechanism 56′ includes a biasing member 170 (FIGS. 7A and 7B), such as a torsional spring, that biases the obstruction member 100′ to the first or HME position. Further, and with specific reference to FIG. 6, the valve mechanism 56′ includes an actuator assembly 172 and a locking device 174. The actuator assembly 172 includes an actuator arm 176 rotatably assembled to, and projecting from, the housing 52′. Rotation of the actuator arm 176 relative to the housing 52′ effectuates transitioning of the obstruction member 100′ (FIGS. 7A and 7B) as described below. Thus, the actuator arm 176 is rotatable from the HME state or position of FIG. 6 to a bypass state or position (i.e., clockwise rotation relative to the orientation of FIG. 6), and vice-versa. In this regard, the locking device 174 is configured to interface with and temporarily lock the actuator arm 176 in the bypass position or state.

For example, in some embodiments, the locking device 174 includes a pair of fingers 178a, 178b projecting from the housing 52′. The fingers 178a, 178b are naturally biased to the orientation reflected in FIG. 6, whereby the fingers 178a, 178b inherently resist deflection toward one another. Further, the fingers 178a, 178b are sized to be selectively captured within an aperture 180 (referenced generally) formed by the actuator arm 176. More particularly, as the actuator arm 176 is rotated toward the fingers 178a, 178b, the fingers 178a, 178b engage within and selectively hold the actuator arm 176 in the bypass position or state due to the natural bias of the fingers 178a, 178b for example. Where desired, the actuator arm 176 can be “released” from the temporary locked state by a user simply lifting up on the actuator arm 176, and rotating the actuator arm 176 away from the fingers 178a, 178b.

With additional reference to FIGS. 7A and 7B, the actuator arm 176 is connected to the obstruction member 100′ via a pin 182 and an attachment body 184. The attachment body 184 connects the pin 182 with the obstruction member 100′ and is further mounted to the actuator arm 176. With this configuration, rotation of the actuator arm 176 is transferred to the obstruction member 100′ via the pin 182 and the attachment body 184. Alternatively, other mechanisms for interconnecting the actuator arm 176 and the obstruction member 100′ are also acceptable.

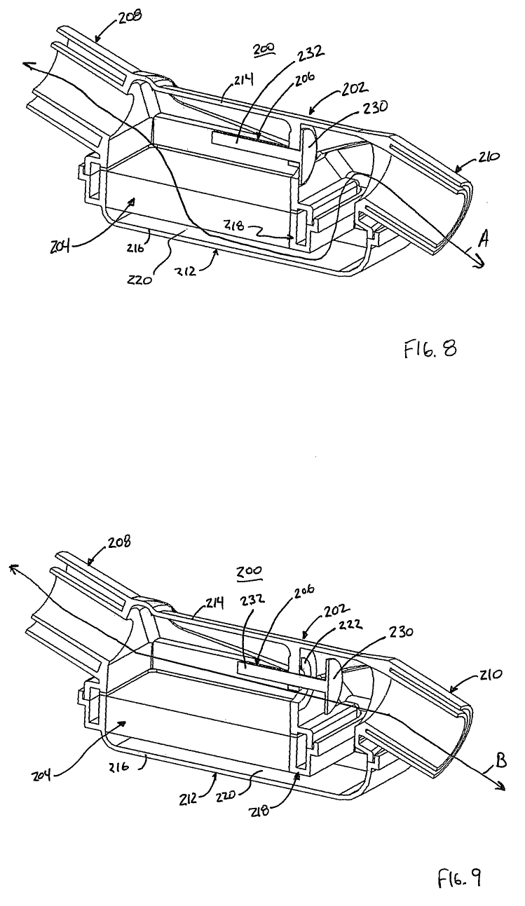

Another embodiment HME unit 200 in accordance with the present disclosure and useful as the HME unit 16 (FIGS. 1 and 2) is partially illustrated in FIGS. 8 and 9. The HME unit 200 is akin to the HME unit 50 (FIG. 3A) described above, and includes a housing 202, an HM media 204, and a valve mechanism 206. The housing 202 forms a first port 208 (e.g., a patient side port), a second port 210 (e.g., ventilator side port), and an intermediate section 212. The HM media 204 can assume any of the forms described above and is retained within the intermediate section 212, with the valve mechanism 206 operating to dictate a pathway through which airflow at least primarily progresses between the first and second ports 208, 210 as described below.

The housing 202, and in particular the intermediate section 212, includes opposing exterior wall segments 214, 216, as well as at least one interior partition 218. The interior partition 218 is spaced from the lower wall segment 216, thereby establishing a gap 220. Further, the interior partition 218 forms an aperture 222 adjacent the upper wall segment 214 with which the valve mechanism 206 is associated as described below. With this construction, then, the housing 202 defines first and second flow paths between the ports 208, 210, as designated by an arrow A in FIG. 8 and an arrow B in FIG. 9. The first flow path A includes the HM media 204, whereas the second flow path B does not. In other words, air flowing through the first flow path A interacts with the HM media 204, and thus constitutes an HME pathway. Conversely, air flowing in the second flow path B does not intimately interact with the HM media 204, and thus serves as a bypass pathway. As with previous embodiments, the second flow path/bypass pathway B is around (e.g., to the side of) the HM media 204.

The valve mechanism 206 includes an obstruction member 230 as described above, for example a valve plate, that is movably assembled within the housing 202. The obstruction member 230 is sized and shaped to selectively encompass or close the aperture 222, with the valve mechanism 206 further including, in some embodiments, a stem 232 that movably associates the obstruction member 230 with the interior partition 218, and in particular the aperture 222. Thus, the obstruction member 230 is transitionable between a first or HME position (FIG. 8) and a second or bypass position (FIG. 9). In the HME position, the obstruction member 230 nests against the interior partition 218, thereby closing the second flow path B. In other words, in the HME position, only the first flow path A is “open” between the first and second ports 208, 210, thereby dictating that airflow through the HME unit 200 must interface with the HM media 204. Conversely, in the second, bypass position, the obstruction member 230 is spaced from the interior partition 218, such that airflow can occur through the aperture 228. Thus, in the bypass position, the second flow path B is open, allowing airflow directly between the first and second ports 208, 210 apart from, or around, the HM media 204.

The positions of FIGS. 8 and 9 represent opposite, maximum points of travel of the obstruction member 230 relative to the housing 202. While the HME unit 200 can be modified to provide a maximum point of travel differing from that of FIG. 9, the so-generated maximum point of travel (or any intermediate position of the obstruction member 230) does not result in the valve plate 230 completely obstructing or closing the first flow path A.

Though not shown, the valve mechanism 206 can include one or more additional features allowing a user to direct the obstruction member 230 to the position corresponding with a desired mode of operation (i.e., HME mode or bypass mode). For example, the valve mechanism 206 can include a biasing device and/or an actuator arm as describe above. Alternatively, any other mechanism (mechanical, electric pneumatic, and/or electrical in nature) can be employed.

In instances where the breathing circuit (not shown) to which the HME unit 200 is assembled is not providing aerosolized medication, the HME unit 200 is operated in the HME mode whereby the obstruction member 230 is placed in the first position (FIG. 8), closing the second flow path B. Thus, airflow through the HME unit 200 (between the ports 208, 210) interacts with the HM media 204, with the HME unit 200 acting like a typical HME unit by the HM media 204 absorbing moisture and heat from patient exhaled air, and transferring the moisture and heat to the inhaled air provided to the patient.

Conversely, where the breathing circuit to which the HME unit 200 is fluidly connected is operating to provide nebulized medication to the patient, the HME unit 200 is transitioned to a bypass mode (FIG. 9) in which the obstruction member 230 is spaced away from the interior partition 218/aperture 222. While the first flow path A remains “open” in the bypass position or mode of the obstruction member 230, a vast majority of airflow through the HME unit 200 will occur along the second flow path B. More particularly, and as described above, the HM media 204 presents a resistance to airflow; because airflow will seek the path of least resistance, in the bypass mode, a vast majority of the airflow between the ports 208, 210 will occur directly along the second flow path B.

Though not shown, the HME unit 200 can incorporate one or more of the additional, optional features described above with respect to the HME unit 50 (FIG. 3A). Similarly, other optional features described below, such as a check valve, can be included.

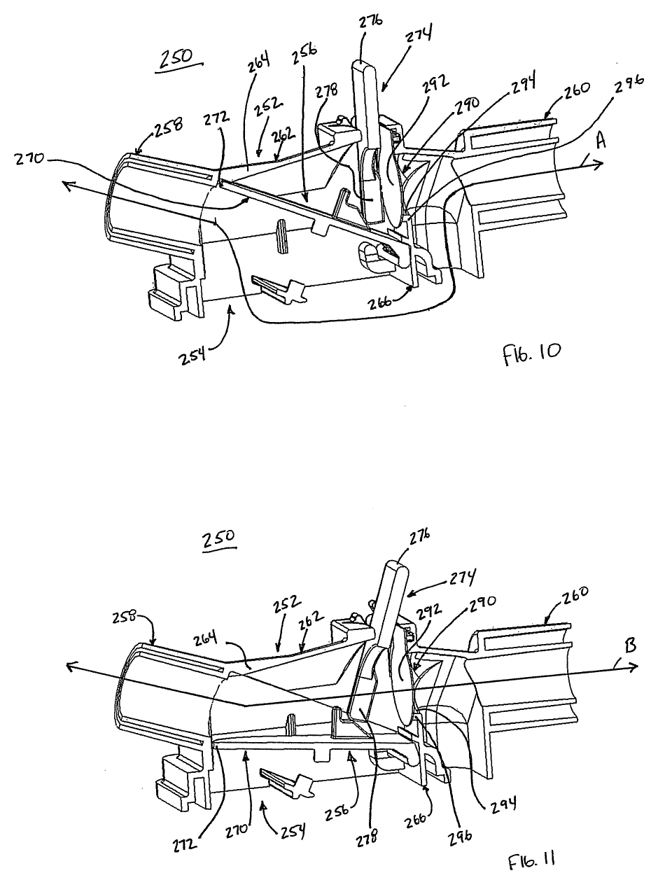

Yet another embodiment HME unit 250 in accordance with principles of the present disclosure and useful as the HME unit 16 (FIGS. 1 and 2) is partially shown in FIGS. 10 and 11. Once again, the HME unit 250 includes a housing 252, an HM media 254 (omitted from the views of FIGS. 10 and 11, but a location of which is generally indicated), and a valve mechanism 256. The housing 252 forms first and second ports 258, 260 extending from opposite sides of an intermediate section 262. The HM media 254 is disposed within the intermediate section 262, with the valve mechanism 256 dictating a pathway through which airflow between the ports 258, 260 is at least primarily directed.

The housing 252 includes exterior wall segments 264, and at least one interior partition 266. The interior partition 266 is spaced from the exterior wall segments 264, thereby defining a first flow path A (FIG. 10) and a second flow path B (FIG. 11). As with previous embodiments, the first flow path A includes the HM media 254, whereas the second flow path B does not. Thus, the first flow path A is an HME pathway, and the second flow path B is a bypass pathway. As with other embodiments, the second flow path/bypass pathway B is around (e.g., to the side of) the HM media 254.

The valve mechanism 256 includes an obstruction member (e.g., a valve plate) 270 movably assembled within the housing 252 and configured to selectively close the second flow path B. More particularly, in a first or HME position (FIG. 10) of the obstruction member 270, a leading end 272 of the obstruction member 270 contacts the exterior wall segment 264, thereby “closing” the second flow path B relative to the first and second ports 258, 260. Thus, in the HME position, the obstruction member 270 directs all airflow between the ports 258, 260 to occur only along the first flow path A.

Conversely, in a second or bypass position of the obstruction member 270, the leading end 272 is transitioned away from the exterior wall segment 264, thereby opening (relative to the obstruction member 270) the second flow path B. In the bypass position, the obstruction member 270 does not effectuate closure of the first flow path A, such that in a bypass mode of the HME unit 250, airflow through the HM media 254 can occur. However, and as previously described, the HM media 254 presents a resistance to airflow, such that in the bypass mode, airflow will seek the path of least resistance and thus primarily occur along the second flow path B.

The positions of FIGS. 10 and 11 represent opposite, maximum points of travel of the obstruction member 270 relative to the housing 252. While the HME unit 250 can be modified to provide a second maximum point of travel differing from that of FIG. 11, the so-generated maximum point of travel (or any intermediate position of the obstruction member 270) does not result in the obstruction member 270 completely obstructing or closing the first flow path A.

Transitioning of the obstruction member 270 by a user between the first and second positions can be facilitated in a number of manners. With some constructions, the valve mechanism 256 includes a biasing device (not shown), such as a spring, that biases the obstruction member 270 to the first or HME position (FIG. 10). An actuator arm 274 is pivotably assembled to the housing 252, and defines first and second ends 276, 278. The first end 276 extends exteriorly from the housing 252, whereas the second end 278 bears against the obstruction member 270. With this but one acceptable construction, then, the obstruction member 270 can be transitioned by a user from the HME position (FIG. 10) to the bypass position (FIG. 11) by applying a rotational or moment force onto the first end 276. Rotation of the actuator arm 274, in turn, causes the second end 278 to bear against and cause movement of the obstruction member 270 in a cam-like fashion. Rotation of the actuator arm 274 in an opposite direction removes the force applied by the actuator arm 274, thus allowing the biasing device to force the obstruction member 270 back to the HME position. Alternatively, a wide variety of other components can be employed to allow a user to select the desired position or mode of operation.

In addition to the above, FIGS. 10 and 11 illustrate an optional check valve feature provided with the HME unit 250. As a point of reference, the check valve feature described below is equally applicable with any of the other HME unit embodiments set forth in the present disclosure. With this in mind, the check valve feature includes a check valve mechanism 290 having a check valve plate 292. The check valve plate 292 is assembled within the housing 252 so as to selectively close the second flow path B.

For example, with some constructions, the housing 252 forms an aperture 294 located between the first and second ports 258, 260 along the second flow path B, and defined by a perimeter 296. The check valve plate 292 is sized and shaped in accordance with a size and shape of the aperture 294, such that when positioned against the perimeter 296, the check valve plate 292 closes the aperture 294. In this regard, the check valve plate 292 is positioned and assembled so as to freely move away from the aperture 294 in the presence of gas flow in a first direction of flow along the flow path B, and close against the aperture 294 in the presence of gas flow in an opposite flow direction. For example, and with specific reference to FIG. 9, airflow along the second flow path B in a flow direction from the second port 260 to the first port 258 causes the check valve plate 292 to pivot away from the aperture 294, thereby permitting airflow along the second flow path B to freely occur. Conversely, airflow along the second flow path B in a flow direction from the first port 258 to the second port 260 forces the check valve plate 292 into engagement with the perimeter 296, thereby closing the aperture 294. Thus, even with the obstruction member 270 in the bypass position of FIG. 11, the check valve plate 292 periodically closes the second flow path B (i.e., only in the presence of gas flow from the first port 258 to the second port 260), such that airflow occurs in this direction only along the first flow path A.

The optional check valve mechanism 290 described above can, in some embodiments, enhance performance of the HME unit 250. For example, during use, the HME unit 250 can be assembled to the patient breathing circuit (not shown), such that the first port 258 serves as a patient side port, whereas the second port 260 serves as a ventilator side port. With these designations in mind, and with the HME unit 250 in the bypass mode, medication droplet-entrained airflow from the ventilator side port 260 to the patient side port 258 occurs primarily along the second flow path B. That is to say, the obstruction member 270 and the check valve plate 292 do not obstruct airflow from the ventilator side port 260 to the patient side port 258. As such, with patient inhalation, the medication droplets are delivered to the patient's lungs and do not overtly contact the HM media 254. With patient exhalation, however, the airflow direction changes (i.e., travels from the patient side port 258 to the ventilator side port 260), thus causing the valve plate 292 to close the aperture 294 as described above. The exhaled air is thus forced to progress through the HM media 254 at which heat and moisture is captured and retained. Because the exhaled air from the patient includes minimal, if any, medication droplets, any clogging concerns of the HM media 254 are greatly minimized.

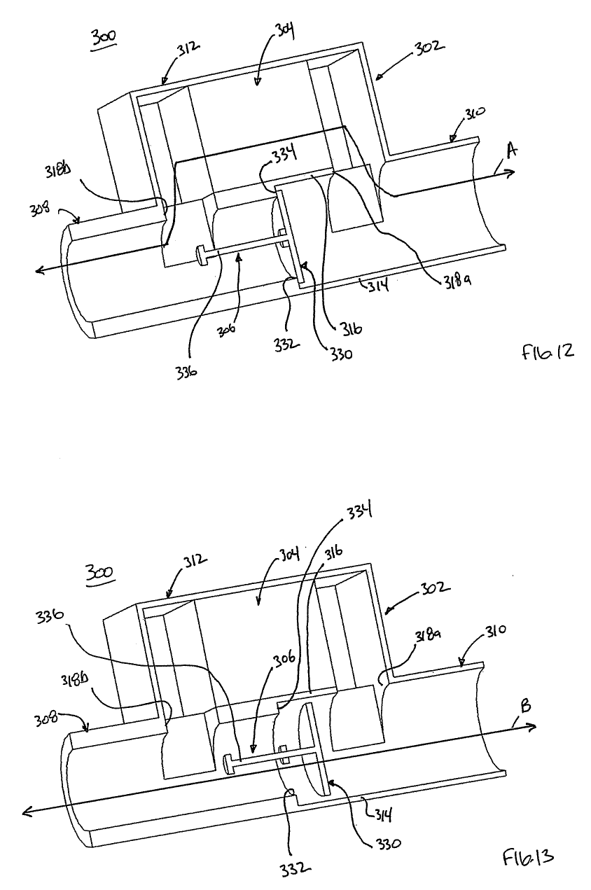

Yet another embodiment HME unit 300 in accordance with aspects of the present disclosure and useful as the HME unit 16 (FIGS. 1 and 2) described above is shown in FIGS. 12 and 13. The HME unit 300 includes a housing 302, an HM media 304, and a valve mechanism 306. The housing 302 defines first and second ports 308, 310, and an intermediate section 312 extending therebetween. The HM media 304 is retained within the intermediate section 312, with the valve mechanism 306 operating to dictate a flow path along which airflow between the ports 308, 310 will at least primarily occur.

The intermediate section 312 includes exterior wall segments 314 and at least one interior partition 316. The interior partition 316 is spaced from other components of the housing 302 to establish a first flow path A and a second flow path B. For example, the interior partition 316 can establish, in part, passages 318a, 318b through which airflow along the first flow path A can occur. Regardless, the HM media 304 is disposed along the first flow path A. Conversely, the second flow path B is apart from (e.g., around or to the side of) the HM media 304. Thus, the first flow path A constitutes an HME pathway, and the second flow path B is a bypass pathway.

The valve mechanism 306 can assume a variety of forms, are in some embodiments includes an obstruction member (e.g., a valve plate) 330 movably assembled within the housing 302. More particularly, the obstruction member 330 is movably positioned so as to selectively “close” the second flow path B. For example, the housing 302 can form an aperture 332 along the second flow path B defined by a wall perimeter 334. The obstruction member 330 is sized and shaped in accordance with a size and shape of the aperture 332 such that in a first or HME position (FIG. 12), the obstruction member 330 abuts against the wall perimeter 334, closing the aperture 332 and thus the second flow path B. Conversely, in a second or bypass position of the obstruction member 330 (FIG. 13), the obstruction member 330 is spaced away from the aperture 332/wall perimeter 334, such that the obstruction member 330 does not obstruct the aperture 332 and thus the second flow path B. In the bypass position, then, airflow between the first and second ports 308, 310 can occur directly along the second flow path B. As with previous embodiments, in the bypass position, the obstruction member 330 does not overtly obstruct the first flow path A such that in the bypass mode, airflow along the first flow path A, and thus to the HM media 304, can occur. However, due to the resistance presented by the HM media 304, airflow will follow the path of least resistance, such that in the bypass position (FIG. 13), airflow at least primarily occurs along the second flow path B.

The positions of FIGS. 12 and 13 represent opposite, maximum points of travel of the obstruction member 330 relative to the housing 302. While the HME unit 300 can be modified to provide a second maximum point of travel differing from that of FIG. 13, the so-generated maximum point of travel (or any intermediate position of the obstruction member 330) does not result in the obstruction member 330 completely obstructing or closing the first flow path A.

As with previous embodiments, the valve mechanism 306 can assume a variety of forms and incorporate various features to effectuate transitioning of the obstruction member 330 between the first and second (or HME and bypass) positions. For example, in some embodiments, the valve mechanism 306 includes a stem 336 that slidably maintains the obstruction member 330 relative to the aperture 332. Further, other components, such as a spring (not shown), and external actuator (not shown), etc., can be provided that afford a user the ability to select the desired position of the obstruction member 330, and thus the operational mode of the HME unit 300.

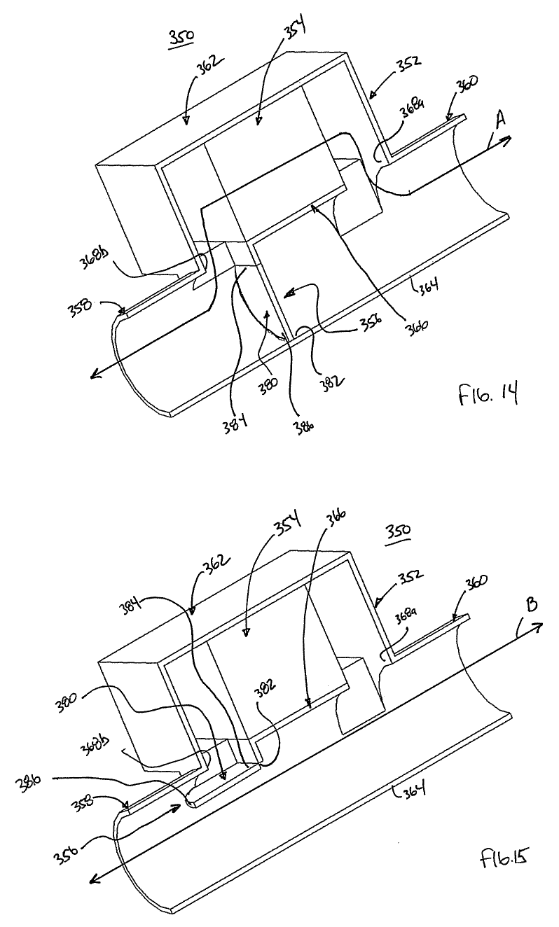

Yet another embodiment HME unit in accordance with principles of the present disclosure and useful as the HME unit 16 (FIGS. 1 and 2) is shown in FIGS. 14 and 15. The HME unit 350 is akin to the HME unit 300 (FIGS. 12 and 13) described above, and includes a housing 352, an HM media 354, and a valve mechanism 356. The housing 352 defines or forms first and second ports 358, 360 extending from opposite sides of an intermediate section 362. The HM media 354 is disposed within the intermediate section 362. The valve mechanism 356 operates to dictate a flow path along which airflow between the first and second ports 358, 360 will at least primarily occur.

The housing 352 includes exterior wall segments 364 and at least one interior partition 366. The interior partition 366 is spaced from other components (e.g., the exterior wall segments 364) to define first and second flow paths A and B. For example, the interior partition 366 can partially establish passages 368a, 368b in establishing the first flow path A. Regardless, the HM media 354 is located along the first flow path A, whereas the second flow path B is apart from (e.g., around or to the side of) the HM media 354. Thus, the first flow path A constitutes an HME pathway, and the second flow path B is a bypass pathway.

The valve mechanism 356 can assume a variety of forms capable of dictating an open or closed state of the second flow path B. For example, in some embodiments, the valve mechanism 356 includes an obstruction member (e.g., a valve plate) 380 positioned to selectively close an aperture 382 formed by the housing 352 along the second flow path B (e.g., between the partition 366 and a corresponding wall segment 364). In a first or HME position of the obstruction member 380 (FIG. 14), the obstruction member 380 encompasses or closes the aperture 382, thereby obstructing the second flow path B. Thus, in the HME position (or HME mode of operation), airflow between the first and second ports 358, 360 occurs only along the first flow path A (and thus must pass through the HM media 354). Conversely, in a second or bypass position of the obstruction member 380 (FIG. 15), the obstruction member 380 is moved away from the aperture 382, such that the second flow path B is no longer obstructed by the obstruction member 380. As with previous embodiments, however in the second or bypass position, the first flow path A is not fully obstructed by the obstruction member 380 such that airflow can occur along both of the flow paths A, B. However, in the bypass position (or bypass mode of the HME unit 350), the HM media 354 presents a resistance to airflow; because airflow will seek the path of least resistance, then, airflow between the ports 358, 360 will occur primarily along the second flow path B.

The positions of FIGS. 14 and 15 represent opposite, maximum points of travel of the obstruction member 380 relative to the housing 302. While the HME unit 350 can be modified to provide a maximum point of travel differing from that of FIG. 15, the so-generated maximum point of travel (or any intermediate position of the obstruction member 380) does not result in the obstruction member 380 completely obstructing or closing the first flow path A.

In some embodiments, the valve mechanism 356 is configured to provide a check valve-like feature. In particular, the valve mechanism 356 includes additional components (not shown) that selectively act upon the obstruction member 380. As a point of reference, the obstruction member 380 can be described as including a pivot end 384 and a free end 386. Movement of the obstruction member 380 relative to the aperture 382 includes the obstruction member 380 pivoting at the pivot end 384. With these conventions in mind, components of the valve mechanism 356 can operate such that in an HME mode of operation, the free end 386 is fixed or locked in the first position of FIG. 14. As described above, the first or HME position of the obstruction member 380 results in airflow through the HME unit 350 occurring only along the first flow path A. In a bypass mode of operation, the components of the valve mechanism 356 release the obstruction member 380 relative to the aperture 382, such that the obstruction member 380 can freely pivot at the pivot end 384. Where the HME unit 350 is assembled to the patient breathing circuit such that first port 358 serves as a patient side port and the second port 360 serves as a ventilator side port, in the bypass mode of the valve mechanism 356, the obstruction member 380 freely pivots away from the aperture 382 with patient inhalation (i.e., gas flow in a direction from the second port 360 to the first port 358). Thus, the obstruction member 380 minimally interferes with the delivery of aerosolized medication to the patient as the patient inhales. In other words, with airflow in a flow direction from the ventilator side port 360 to the patient side port 358, the obstruction member 380 opens the second flow path B. With patient exhalation (i.e., flow direction from the patient side port 358 to the ventilator side port 360), the airflow forces the obstruction member 380 back to the closed position relative to the aperture 382, thereby forcing exhaled air through the HM media 354 (and an optional filter (not shown)). On the next patient inhalation, the obstruction member 380 freely opens again, etc.

Regardless of an exact design, the HME unit of the present disclosure provides a marked improvement over previous designs. The HME unit provides viable HME and bypass operational modes. However, unlike conventional bypass-type HME unit designs, the HME unit of the present disclosure is compact and streamlined, and user transitioning between the HME and bypass modes is easily accomplished (e.g., frictionally fitted housing components are not required to be rotated relative to one another). Further, the HME unit is relatively inexpensive to manufacture, and is easily adapted to incorporate additional features such as filters, etc.

Although the present disclosure has been described with reference to preferred embodiments, workers skilled in the art will recognize that changes can be made in form and detail without departing from the spirit and scope of the present disclosure.

What is claimed is:

1. A heat and moisture exchange (HME) unit comprising:
a housing forming a first port, a second port, and an intermediate section extending between the first and second ports, the intermediate section defining first and second flow paths fluidly connecting the first and second ports;
a heat and moisture retaining media (HM media) maintained within the intermediate section along the first flow path; and
a valve mechanism including an obstruction member movably retained within the housing and transitionable between opposing, first and second maximum points of travel;
wherein the HME unit is configured such that:
at the first maximum point of travel, the obstruction member closes the second flow path to direct airflow through only the first flow path,
at the second maximum point of travel, the obstruction member permits airflow through both of the first and second flow paths.

2. The HME unit of claim 1, wherein the HM media includes opposing, first and second major surfaces and is located within the housing such that the first major surface fluidly faces the first port and the second major surface fluidly faces the second port, and further wherein, in any position of the obstruction member, the obstruction member does not completely obstruct fluid communication between the first major surface and the first port, or between the second major surface and the second port.

3. The HME unit of claim 1, wherein the HM media has a length not less than a width, and the width greater than a thickness, and further wherein the HM media is arranged such that the first flow path is through the thickness of the HM media.

4. The HME unit of claim 1, wherein the HM media has opposing, first and second major surfaces separated by minor side surfaces, and further wherein the HM media is arranged relative to the first flow path such that the first major surface fluidly faces the first port and the second major surface fluidly faces the second port.

5. The HME unit of claim 4, wherein the housing includes at least one exterior wall and at least one interior partition combining to define at least a portion of the first flow path and further wherein the first and second major surfaces are spaced from the walls of the housing.

6. The HME unit of claim 5, wherein each of the minor side surfaces of the HM media abuts at least one of the walls of the housing.

7. The HME unit of claim 5, wherein the first flow path is U-shaped in cross-section.

8. The HME unit of claim 7, wherein the U-shaped first flow path is defined, at least in part, by a bottom wall of the housing, and further wherein the HM media is spaced from the bottom wall.

9. The HME unit of claim 1, wherein the first and second flow paths each include passageway opening adjacent the first port with the obstruction member being disposed proximate the passageway openings, and further wherein a size of the obstruction member is greater than a size of the passageway opening of the second flow path and is less than a size of the passageway opening of the first flow path.

10. The HME unit of claim 1, wherein the valve mechanism includes a spring biasing the obstruction member to the first maximum point of travel.

11. The HME unit of claim 10, wherein the valve mechanism further includes an actuator arm accessible from an exterior of the housing and connected to the obstruction member, the actuator arm configured to transition the obstruction member from the first maximum point of travel to the second maximum point of travel.

12. The HME unit of claim 11, wherein the valve mechanism further includes a release switch configured to selectively engage the actuator arm upon transitioning of the obstruction member to the second maximum point of travel.

13. The HME unit of claim 12, wherein the release switch is accessible from an exterior of the housing and is configured to release the actuator arm in response to a user-applied force.

14. The HME unit of claim 1, wherein the HM media defines a major plane, and further wherein upon final assembly, a central axis of the second port is substantially parallel with the major plane and a central axis of the first port is non-parallel relative to the major plane.

15. The HME unit of claim 1, further comprising:
a check valve mechanism including a check valve plate movably retained within the housing along the second flow path;
wherein the check valve mechanism is configured such that the check valve plate permits airflow through the second flow path in a first flow direction and prevents airflow through the second flow path in a second, opposite flow direction.

16. The HME unit of claim 15, wherein the check valve plate is spaced from the obstruction member.

17. The HME unit of claim 16, wherein the check valve mechanism is configured to lock the check valve plate in an HME mode of operation.

18. The HME unit of claim 1, further comprising:
a secondary filter disposed within the intermediate section adjacent the HM media.

19. The HME unit of claim 18, wherein the secondary filter is maintained along the first flow path.

20. The HME unit of claim 1, wherein the intermediate section includes an upper segment and a lower segment, and further wherein each of the ports extend from the upper segment, and the HM media is disposed within the lower segment.

21. A heat and moisture exchanger (HME) unit comprising:
a housing forming a first port, a second port, and an intermediate section extending between the first and second ports;
a heat and moisture retaining media (HM media) defining opposing, first and second major surfaces, wherein the HM media is disposed within the housing such that the first major surface fluidly faces the first port and the second major surface fluidly faces the second port; and
a valve mechanism including an obstruction member movably assembled within the intermediate section fluidly between the first major surface of the HM media and the first port, the obstruction member being transitionable from an HME position in which the obstruction member completes an HME flow path from the first port, through the HM media, and to the second port and closes a bypass flow path around the HM media;
wherein the HME unit is configured such that in any position in the obstruction member relative to the housing, at least a portion of the first major surface of the HM media remains fluidly open to the first port.

22. A heat and moisture exchanger (HME) unit comprising:
a housing forming a first port, a second port, and an intermediate section extending between the first and second ports, the intermediate section defining first and second flow paths fluidly connecting the first and second ports;
a heat and moisture retaining media (HM media) maintained within the intermediate section along the first flow path;
a single, secondary filter maintained within the intermediate section along the first flow path;
wherein the HM media and the secondary filter are apart from the second flow path; and
a valve mechanism including an obstruction member movably assembled within the housing and transitionable between a first position in which the first flow path is open and the second flow path is closed, and a second position in which at least the second flow path is open.

23. The HME unit of claim 22, wherein the HM media defines a plurality of exterior faces at least one of which has a largest surface area as compared to remaining ones of the plurality of exterior faces, and further wherein a surface area of an exterior face of the secondary filter approximates an area of the exterior face of the HM media defining the largest surface area.
